(19) United States
(12) Patent Application Publication
(10) Pub. No.: US 2012/0283269 A1
(43) Pub. Date: Nov. 8, 2012

(54) Method and Compositions for Suppression of Aging

(75) Inventors: Mikhail V. Blagosklonny, Depew, NY (US); Andrei V. Gudkov, East Aurora, NY (US); Zoya N. Demidenko, Orchard Park, NY (US)

(73) Assignee: HEALTH RESEARCH INC., Buffalo, NY (US)

(21) Appl. No.: 13/505,573

(22) PCT Filed: Nov. 4, 2010
(86) PCT No.: PCT/US2010/055432

§ 371 Date: Jul. 20, 2012

Related U.S. Application Data
(60) Provisional application No. 61/258,106, filed on Nov. 4, 2009.

(51) Int. Cl. A61K 31/496; A61K 31/436; A61P 9/00; A61P 9/12; A61P 19/02; A61P 25/28; A61P 3/10; A61P 25/16; A61P 29/00; C12N 5/071; A61P 19/10

(52) U.S. Cl. 514/254.07; 435/375; 514/291

(57) ABSTRACT

The present invention provides a method of suppression and/or deceleration of mammalian cellular aging. The method involves contacting mammalian cells with a composition that contains a non-genotoxic inducer of p53 (NGIP). In certain embodiments, the NCIP is a Mdm-binding agent or Mdm-2 antagonist. The NGIP can be nutlin, nutlin-3A, a nutlin analog, or a combination thereof. The invention also provides a method for reducing cellular hypertrophy in an organism by administering a composition that contains an anti-hypertrophic compound, such as nutlin, nutlin-3A, a nutlin analog, rapamycin or a rapamycin analog and combinations thereof, to the organism.

This application claims priority to U.S. provisional application No. 61/258,106, filed Nov. 4, 2010, the disclosure of which is incorporated herein by reference.

### BACKGROUND OF THE INVENTION

It is estimated that in the next 25 years, the number of individuals over the age of 65 in the United States will at least double, and the populations of elderly individuals in many other countries are growing at even faster rates.

With increased chronological age, there is a dramatically increased risk of numerous debilitating diseases. Therefore, there is an ongoing need to identify strategies to prevent, delay or treat age-associated diseases. The present invention addresses this need.

### SUMMARY OF THE INVENTION

The present invention provides a method of suppression and/or deceleration of mammalian cellular aging. The method comprises contacting mammalian cells with a composition comprising a non-genotoxic inducer of p53 (NGIP). In certain embodiments, the NCIP is a Mdm-binding agent or Mdm-2 antagonist. In certain embodiments, the NGIP can be nutlin, nutlin-3A, a nutlin analog, or a combination thereof.

The method is expected to be suitable for prophylaxis and/or therapy of age-related diseases and/or cellular hypertrophy in any individual. In on embodiment, an individual treated according to the method of the invention has not been diagnosed with cancer. In other embodiments, the invention provides a method for reducing cellular hypertrophy in an organism by administering a therapeutically effective amount of a composition comprising an anti-hypertrophic compound to the organism. Non-limiting examples of anti-hypertrophic compounds that can be used in performance of the invention include nutlin, nutlin-3A, a nutlin analog, rapamycin or a rapamycin analog and combinations thereof.

In various embodiments, the method of the invention results in suppression and/or deceleration of mammalian cellular aging. The suppression and/or deceleration of mammalian cellular aging can comprise mammalian cells becoming quiescent.

### BRIEF DESCRIPTION OF THE FIGURES

FIG. 1. Nutlin-3a converted senescence into quiescence. a. HT-p21-9 cells were treated with IPTG, 10 μM nutlin-3a and IPTG plus nutlin-3a for 3 days. Cells were stained for beta-Gal and photographed (original magnification ×400). Bar scale—50 μm. b. HT-p21-9 cells were treated with IPTG, 10 μM nutlin-3a and IPTG plus nutlin-3a for 3 days. After 3 days, cells were washed to remove IPTG and nutlin-3a. Then cells were cultured in fresh medium until colonies become visible. Dishes were stained with crystal violet and photographed on day 4 (control) or on day 9 (IPTG- and nutlin-3a-treated).c. HT-p21-9 cells were treated with IPTG in the presence or absence of 10 μM nutlin-3a for 3 days. Before wash. Live HT-p21-9 cells (expressing GFP for better visualization of live cells) were photographed (original magnification ×100) under blue light. 3 d wash. Three days after drug removal. 9 d wash. Nine days after drug removal, cells were stained with crystal violet and photographed. d. Nutlin-3a dose response. HT-p21-9 cells were plated with IPTG and 0, 2.5, 5, or 10 μM nutlin-3a. After 3 days, the plates were washed and cells were incubated for an additional 9 days in fresh medium, stained with crystal violet and photographed. e. Colonies per dish. HT-p21-9 cells were plated with IPTG and 0, 1.2, 2.5, 5, 10 or 20 μM nutlin-3a, as indicated. After 3 days, the plates were washed and cells were incubated for an additional 9 days in fresh medium. Colonies were counted and results are shown as percent of control (IPTG alone). f. Cells per dish. As in panel e. Cells were trypsinized and counted. Results are shown as percent of control (IPTG alone).

FIG. 2. p53-dependent effects of nutlin-3a. a. HT-p21-GSE56 and HT-p21-9 cells were treated with IPTG alone (0) or IPTG plus rapamycin (R) and nutlin-3a (N). Control cells were left untreated (no IPTG). After 1 day, cells were lysed and immunoblot was performed. b. HT-p21-GSE56 (open circles) and HT-p21-9 cells (closed circles) were treated with nutlin-3a for 5 days and then counted. As a negative control, parental cells were treated with nutlin-3b (open squares).c-d. HT-p21-GSE56 and HT-p21-9 cells were treated with IPTG alone or with IPTG+rapamycin (I+R) or IPTG+nutlin-3a (I+N), as indicated. Control cells were left untreated (no IPTG). c. Morphology. After 3 days, cells were stained for beta-Gal. Scale bars—50 μm.d. Colony formation. After 3 days, cells were washed and incubated in fresh medium w/o drugs for an additional 9 days. Plates were stained with crystal violet and photographed. e. Proliferative potential (PP). After 3 days, HT-p21-GSE56 cells were washed and incubated in fresh medium w/o drugs. Cells were counted and results are shown as percent of IPTG alone.

FIG. 3. Effects of nutlin-3a on the mTOR pathway and protein synthesis. a. Immunoblot. HT-p21 cells were treated with IPTG alone or with IPTG plus 500 nM rapamycin (R), 25 μM LY-294002 (L), 10 μM U0126 (U) or 10 μM nutlin-3a (N) for 24 hr. Immunoblot was performed as described in the methods for Example 1 below. b. Immunoblot. HT-p21 cells were treated rapamycin (R) and nutlin-3a (N) in the presence or absence of IPTG for 18 hr. Immunoblot was performed as described in Methods. c. Effects of nutlin-3a on PP (proliferative potential) of IPTG-treated HT-p21-9 cells in the absence (black bars) or presence (open bars) of rapamycin (500 nM). After 3 days, cells were washed and incubated in fresh medium w/o drugs for an additional 7 days. Cells were counted and are shown as percent of IPTG alone. d. Effects of nutlin-3a and rapamycin on cellular hypertrophy caused by IPTG. Cells were treated with either IPTG alone (black bars) or IPTG plus rapamycin (white bars) or plus nutlin-3a (grey bars). On days 2, 3, 4, and 5 cells were lysed and protein content per well was measured. The numbers presented correspond to protein content per cell, since the cells did not proliferate and their numbers were unchanged during the course of the experiment. e. Effects of nutlin-3a on protein synthesis ([³⁵S]methionine/cysteine incorporation). Cells were labeled with [³⁵S]methionine/cysteine as described in Methods for Example 1.

FIG. 4. Effects of ectopic and endogenous p53 on senescence in HT-p21-9 and WI-38-tert. a. p53-expressing adenovirus (Ad-p53) suppresses senescent morphology caused by IPTG in HT-p21-a cells. HT-p21-a cells were treated with IPTG and infected with Ad-p53. After 3 days, cells were photographed (original magnification ×200): Upper panel. Under blue light to visualize cells expressing p53 (green cells). Lower panel. Under visible light to visualize all cells. Red arrows indicate cells lacking p53 expression. All of these cells show large, flat cell morphology. Green arrows indicate cells expressing p53. b-d. Effects of nutlin-3a on cellular senescence in WI-38-tert fibroblasts, WI-38-tert cells were treated with 200 μM H₂O₂ for 30 min in serum free medium. Then, the medium was replaced for complete medium (10% serum) with or without 10 μM nutlin-3a. b. After 1 day, cells were lysed and immunoblot was performed as described in Methods for Example 1. c. After 3 days, the cells were washed (nutlin-3a was removed) and grown for 3 additional days in fresh complete medium. Cells were then stained for beta-Gal activity and microphotographed. Scale bar—50 μm.d. After 3 days, the cells were washed (nutlin-3a was removed) and grown for 6 additional days in fresh complete medium. Cells were then trypsinized and counted. In control, cells reached confluence by day 5 and did not proliferate further. Results are shown as percent of control.

FIG. 5. Senescent versus quiescent morphology. HT-p21 cells were treated with IPTG, nutlin-3a (10 μM) and IPTG plus nutlin-3a for 3 days or left untreated (control). Live cells, visualized with GFP (×100). In control, cells underwent 3 divisions, forming micro-colony. IPTG treated cells (large and flat) did not undergo any divisions. Nutlin-3a-treated cells were arrested after one division with normal cell morphology.

FIG. 6. HT-p21-9 cells were plated in 100 mm dishes and treated with IPTG in the presence or absence of nutlin-3a for 3 days. Nine days after drug removal. a. Cell number per dish. Cells per dish were counted. b. Cell number per a colony. Number of cells per colony was calculated. A number of cells per colony was 200-250 (approximately equals to 8 divisions) by day 9. Thus, quiescent cells were characterized by normal proliferative potential after release from IPTG+nutlin-3a.

FIG. 7. Preservation of proliferative potential by Nutlin-3a. a. Comparison of nutlin-3a and nutlin-3b in HT-p21-a cells. HT-p21-a cells were treated with IPTG in the presence of indicated concentrations of nutlin-3a (closed circles) and nutlin-3b (open squares) for 6 days. Then medium was changed and cells were counted after 8 days. b. Comparison of nutlin-3a and nutlin-3b in HT-p16 cells. HT-p16 cells were treated with IPTG in the presence of indicated concentrations of nutlin-3a (closed circles) and nutlin-3b (open squares) for 3 days. Then medium was changed and cells were counted after 5 days.

FIG. 8. Effects of IPTG and 500 nM rapamycin on protein synthesis ([³⁵S]methionine/cysteine incorporation). Cells were treated as indicated for 24 hrs and then labeled with [³⁵S]methionine/cysteine as described in Methods for Example 1.

FIG. 9. a. Effects of Ad-p21 and Ad-p53 on cellular morphology. p16-5 cells, derivatives of HT-1080 cells, were infected with either p21-expressing adenovirus (upper panel: Ad-p21) or p53-expressing adenovirus (lower panel: Ad-p53). Ad-p21 (upper panel) caused large, flat cell morphology. Ad-p53 did not cause large, flat cell morphology. Cells were photographed at ×200. b. Ad-p53 suppresses senescent morphology caused by Ad-p21.p16-5 cells, derivatives of HT-1080 cells, were infected with Ad-p21 and Ad-p53. upper panel. Under blue light to visualize cells expressing p53 (green cells) (×200). lower panel. Under visible light to visualize all cells (×200). Red arrow is pointed at the cell with weak p53 expression. All other cells did not acquire large, flat cell morphology.

FIG. 10. Effects of Ad-p53 on senescent morphology caused by p16. p16-5 cells, derivatives of HT-1080 cells, were treated with IPTG (upper panel) and IPTG plus Ad-p53 (lower panel). IPTG (upper panel) causes large, flat cell morphology. Ad-p53 prevents this morphology. Cells were photographed at visible light and blue light (×200) to visualize cells expressing p53.

FIG. 11. Effects of Ad-p21 and Ad-p53 on senescent morphology in WI-38-tert fibroblasts. WI-38-tert cells were infected with either p21-expressing adenovirus (Ad-p21) or p53-expressing adenovirus (Ad-p53) or both. After 3 days, cells were stained for beta-Gal.

FIG. 12. Effects of nutlin-3a on p53 levels and S6/S6K phosphorylation in WI-38-tert fibroblasts. WI-38-tert cells were treated with indicated concentrations of nutlin-3a and 500 nM rapamycin (Rapa), as indicated, for 24 hr. Immunoblot for p53, p-S6, p-S6K, S6 and actin was performed as described in Methods for Example 1.

FIG. 13. Schema: Suppression of senescence by p53. a. p21 causes cell cycle arrest, leading to senescence b. p53 causes cell cycle arrest and simultaneously inhibits the senescent program, leading to quiescence.

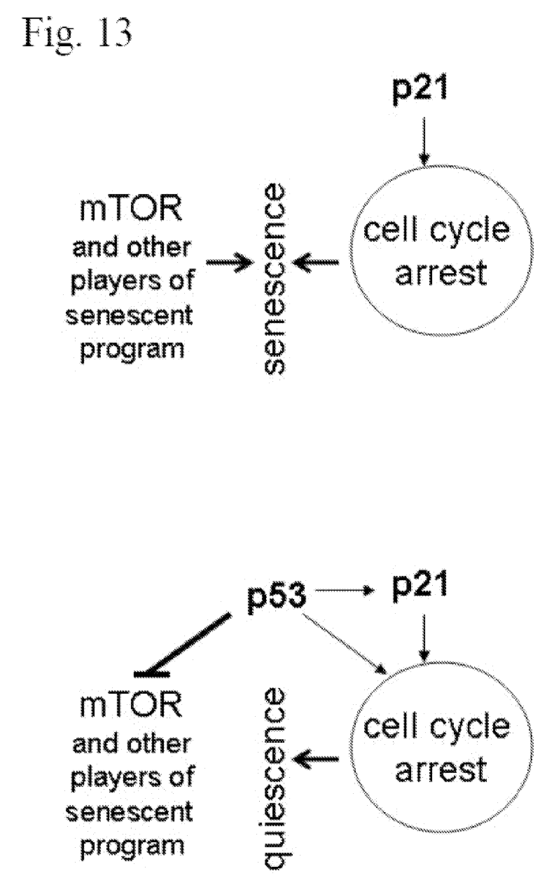

FIG. 14. Inhibition of cell proliferation by IPTG

Closed bars: HT-p21 cells were treated with IPTG (+IPTG). Cells do not proliferate. Open bars: Untreated HT-p21 cells. Exponentially proliferating cells. Cells were counted daily.

FIG. 15. Total cellular mass growth during senescence induction

HT-p21 cells were grown in 60 mm wells and soluble protein and GFP were measured daily. Closed bars: HT-p21 cells were treated with IPTG (+IPTG). Open bars: Untreated HT-p21 cells (−IPTG). In both proliferating (−IPTG) and non-proliferating (+IPTG) conditions, protein per well and GFP per well were increasing. In panel B, protein was measured in duplicate and shown without standard deviations, therefore statistical difference between −IPTG and +IPTG should not be considered. The panel simply illustrates exponential growth in both conditions.

FIG. 16. Cellular hypertrophy during senescence induction

HT-p21 cells were grown in 60 mm wells and cell numbers, soluble protein and GFP were measured daily. Closed bars: HT-p21 cells were treated with IPTG (+IPTG). Open bars: Untreated HT-p21 cells (−IPTG). Protein per cell and GFP per cell were constant in proliferating (−IPTG) cells. Protein per cell and GFP per cell increased exponentially in non-proliferating (+IPTG) cells.

FIG. 17. Visualization of cellular hypertrophy

HT-p21 cells express enhanced green fluorescent protein (GFP) under the constitutive viral CMV promoter. Expression of GFP per cell is a marker of cellular hypertrophy. Low cell density—2 thousand cells were plated in 100 mm dish and treated with either IPTG or IPTG+Rapamacin.

FIG. 18. Correlation between S6 phosphorylation, hypertrophy and loss of proliferative potential in senescent cells. HT-p21 cells were plated in 6 well plates and treated with IPTG plus the increasing concentrations of rapamycin (from 0.16 to 500 nM). At concentration 0, cells were treated with IPTG alone. A. Cellular hypertrophy: protein and GFP. After 3 days, soluble protein and GFP were measured per well. [Note: in non-proliferating cells, protein/well is a measure of protein/cells]. Results are shown as percent of IPTG alone (0) without rapamycin. B. After 3 days, cells were lysed and immunobloted for p-S6, S6 and p21.

C. PC: preservation of proliferative competence. After 3 days, cells were washed to remove IPTG and RAPA. Cells were incubated for additional 5 days in the fresh medium and then were counted. Results are shown as percent of IPTG alone (0) without rapamycin.

FIG. 19. Clonal proliferation of competent cells. HT-p16 cells were plated in 100-mm plates. The next day, 50 μM IPTG with or without rapamycin, if indicated (RAPA), was added. After 3 days, the plates were washed to remove IPTG and RAPA. A. Photographs. Upper panel: On days 5 and 8 (after IPTG removal), plates were fixed, stained and photographed. Lower panel: On days 5 and 8 (after IPTG removal), plates were fixed, stained and photographed. B. Number of colonies. On days 6, 7, 8 and 9 (after IPTG removal), plates were fixed, stained and photographed. The number of colonies was counted and results are shown as percent of plated cells in log-scale.

FIG. 20. The dynamics of cell numbers. 500 HT-p21 cells were plated in 12 well plates. On the next day, either IPTG alone (I) or IPTG plus rapamycin (I+R) were added. After 3 days, plates were washed (I/w and I+R/w) or left unwashed. Cells were counted at days 1, 3, 6 and 9. Upper panel: linear-scale. Lower panel: log-scale. Open and closed squares: IPTG and IPTG plus Rapa, respectively. Open and closed circles: IPTG washed (I/w) and IPTG plus Rapa washed (I+R/w), respectively. In the presence of IPTG (open squares) and IPTG plus rapamycin (closed squares), the cells did not proliferate.

FIG. 21. Loss of hypertrophy during proliferation of competent cells. 500 HT-p21 cells were plated in 12 well plates. The next day, either IPTG alone or IPTG plus rapamycin were added. After 3 days, plates were washed (I/w and I+R/w) or left unwashed. GFP per well was measured and cells were counted at days 1, 3, 6 and 9. GFP per cell was calculated (upper panel). Results are shown in arbitrary units (M±m). Open and closed squares: IPTG and IPTG plus Rapa, respectively. Open and closed circles: IPTG washed (I/w) and IPTG plus Rapa washed (I+R/w), respectively. When cells resumed exponential proliferation, GFP per cell dropped to normal levels. Due to robust proliferation, there was an increase of GFP per well.

FIG. 22. The morphology of cells during recovery. 500 HT-p21 cells were plated in 12 well plates. The next day, IPTG (A) or IPTG plus rapamycin (B) was added. After 3 days, plates were washed and microphotographs were taken after additional 3 days. Cells were stained for beta-Gal. A: I/w; B: FR/w.

FIG. 23. Visualization of loss of hypertrophy during proliferation of competent cells. 500 HT-p21 cells (A) were treated with IPTG (B) or IPTG plus rapamycin (C), as indicated, or left untreated. After 3 days, plates were washed and incubated without drugs to allow proliferation. A. Normal size of proliferating cells. B. Cellular hypertrophy of senescent cells. C. Example 1. Clonal proliferation of competent cells results in loss of hypertrophy. C. Example 2. Cells that remained arrested remained hypertrophic.

FIG. 24. Induction of p21 by IPTG. HT-p21 cells were plated in 6 well plates and treated with IPTG with or without rapamycin as indicated. The next day, cells were lysed and immunoblot for p-S6, S and p21 was performed as described in Methods. IPTG dramatically induced p21, without affecting S6 phosphorylation, whereas rapamycin inhibited S6 phosphorylation, without affecting p21 induction.

FIG. 25. Loss of hypertrophy following release. HT-p21 cells were treated with IPTG plus 500 nM rapamycin for 3 days. Then the cells were washed and the cells were incubated in the fresh medium without drugs. At indicated days, soluble protein, GFP and cell numbers were measured per well. Protein (pr) per cell and GFP per cell were calculated and plotted in arbitrary units.

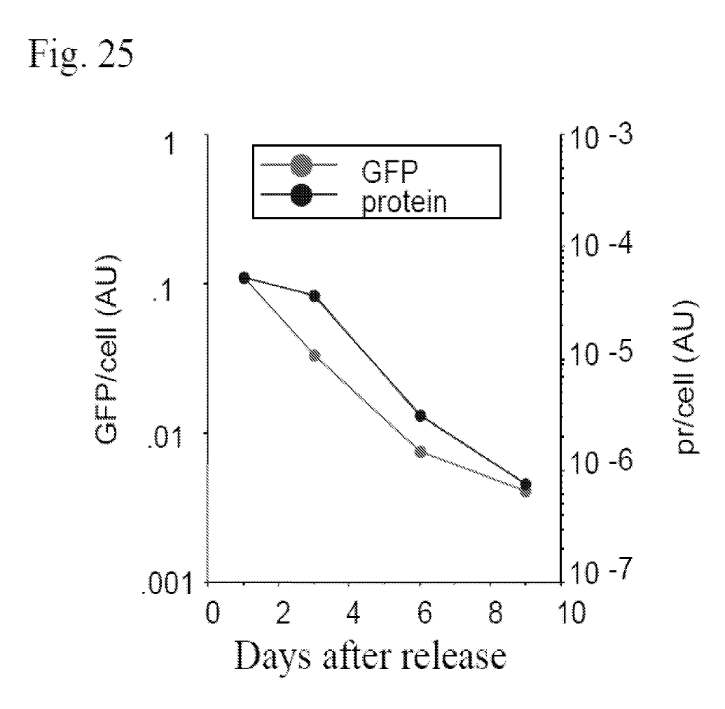

### DESCRIPTION OF THE INVENTION

The present invention provides a method for prophylaxis and/or therapy of age-related diseases and/or symptoms of such diseases. Without intending to be bound by any particular theory, it is considered that the invention achieves these effects by suppressing the aging process.

The present invention takes advantage of the discovery disclosed here for the first time that p53, historically thought of as an emblematic inducer of cellular senescence, instead participates in suppression of cellular senescence. In this regard, in previous studies, suppression of senescence by p53 was apparently masked by p53-induced cell cycle arrest, which (if prolonged) can lead to senescence. Since previous studies relied on p53 itself to cause cell cycle arrest, it was not possible to distinguish whether p53 actively suppressed senescence or merely failed to induce it in some experimental situations. However, in the present invention we are able to differentiate between these two scenarios by testing the effect of p53 on senescence induced by p21 or p16 rather than p53 itself. We discovered that in either p21- or p16-arrested cells, p53 converted senescence (irreversible arrest with senescent morphology) into quiescence (reversible arrest with preservation of proliferation capacity and no senescent morphology). Thus, the invention is based in part on our discovery of paradoxical suppression of cellular senescence by p53.

In connection with the present invention, it is considered that “aging” means organismal aging and/or cellular aging (senescence). Organismal aging results from cellular aging and is considered to be an increase of the probability of death with age (time). Suppression of aging decreases the probability of death and thus increases life span. Organismal aging is manifested by age-related diseases, the incidence of which increases with age. Death from aging means death from age-related diseases. Suppression of aging delays one, some or most age-related diseases. Slow aging is manifested by delayed age-related diseases. Slow aging is considered to be a type healthy aging. Age-related diseases are considered to be biomarkers of organismal aging. A compound that delays age-related diseases extends life span and can be considered an anti-aging drug. Likewise, a compound that suppresses aging delays age-related diseases.

Without intending to be bound by any particular theory, cellular aging (senescence) is considered to be caused by overstimulation and overactivation of signal transduction pathways such as the mTOR pathway, especially when the cell cycle is blocked, leading to cellular hyperactivation and hyperfunction. In turn, this causes secondary signal resistance and compensatory incompetence. Both cellular hyperfunction and signal-resistance cause organ damage (including in distant organs), manifested as aging (subclinical damage) and age-related diseases (clinical damage), eventually leading to organismal death. Non-limiting example of markers of cellular aging are considered to be cellular hypertrophy, permanent loss of proliferative potential, large-flat cell morphology and beta-Gal staining

In performance of the present invention, we have demonstrated that p53 suppresses cellular aging, and that non-genotoxic inducers of p53 (NGIP) prevent, decelerate and suppress cellular aging. Further, cellular aging is characterized not only by permanent loss of proliferative potential, distinct morphology, a hyper-secretory and pro-inflammatory phenotype, but also by large size of the senescent cell (hypertrophy). Hypertrophy of aging cells contributes to age-related diseases such as prostate enlargement, cardiac hypertrophy, renal hypertrophy, arterial wall thickening, and obesity, whereby obesity results from an increase of the size of fat cells and not necessarily not from an increase of cell numbers. We have demonstrated that both NGIPs (such as Nutlin-3A) and inhibitors of mTOR (such as rapamycin) decrease hypertrophy of senescent cells. Thus, it is expected that anti-hypertrophic agents such as nutlin-3a and rapamycin could be used to decrease cell size in age-related diseases, thereby further contributing to anti-aging effects of these compounds.

Results presented here are notable because p53 causes apoptosis, reversible cell cycle arrest (quiescence) and irreversible cell cycle arrest (senescence). It has been assumed that p53 actively causes senescence.

We have demonstrated that nutlin-3A induces quiescence (reversible arrest without senescent morphology) in HT-p21 and WI-38-tert cells. In the same cell lines, inducible ectopic p21 and p16 caused senescence. According to the conventional doctrine, nutlin-3A in previous observations simply failed to activate the senescent program because of, for example, insufficient induction of p21. In contrast, and without intending to be bound by any particular theory, we consider that nutlin-3A inhibits the senescence program. Here we demonstrate that p53 indeed converts senescence into quiescence. We conclude that aside from its ability to induce cell cycle arrest, p53 is a potent aging-suppressor. Thus, for the first time we demonstrate that p53 suppresses cellular senescence which has not been previously appreciated, and exploit this finding via the method of the invention. Further, we demonstrate that ectopic p53 itself suppresses senescence. Thus, it is expected that any p53-inducing agents will also suppress senescence.

In one embodiment, the method comprises contacting a cell or administering to an individual a composition comprising a non-genotoxic inducer of p53 (NGIP), wherein the contacting and/or the administration results in prevention, inhibition or treatment of an age related disease or a symptom of such a disease. The NGIP can be used in an amount effective to prevent, inhibit or treat the age related disease or symptom thereof

In one embodiment, the invention provides a method of suppression and/or deceleration of mammalian cellular aging by contacting the cells with a NGIP. In one embodiment, the mammalian cells are present in a human. In one embodiment, the human has not previously been administered an NGIP.

In one embodiment, an individual for which the method of the invention is performed has not previously been administered an NGIP. In one embodiment, the individual does not have cancer.

In one embodiment, the suppression and/or deceleration of mammalian cellular aging is characterized in that the mammalian cells that are contacted with the NGIP become quiescent. In one embodiment, prior to being coaxed into quiescence by performance of the method of the invention, the mammalian cells are senescent. Thus, in certain embodiments the invention provides methods for coaxing mammalian cells to become quiescent.

Another embodiment of the invention relates to prophylaxis and/or treatment of hypertrophy of aging cells. Hypertrophy of aging cells contributes to age-related diseases such as prostate enlargement, cardiac hypertrophy, renal hypertrophy, arterial wall thickening, and hypertrophic fat cells, or obesity. In this regard, we demonstrate that NGIPs and inhibitors of mTOR decrease hypertrophy of senescent cells. Thus, in one embodiment, the invention comprises a method of inhibiting or reducing hypertrophy of cells by administering to an individual in need thereof a composition comprising an effective amount of an NGIP, an inhibitor of mTOR, or a combination thereof. In various embodiments, the individual to whom the inhibitor of mTOR is administered has not previously received an inhibitor of mTOR, and/or the individual has not received an organ transplantation and/or is not a candidate for organ transplantation. In one embodiment, the individual is not in need of immunosuppression therapy.

It is expected that the method of the invention could be used for prophylaxis or therapy of any age-related diseases and/or cellular hypertrophy in any individual. Non-limiting examples of age-related diseases include benign tumors, cardiovascular diseases (such as stroke, atherosclerosis, hypertension), angioma, osteoporosis, insulin-resistance and type II diabetes (diabetic retinopathy, neuropathy), Alzheimer's disease, Parkinson's disease, age-related macular degeneration, arthritis, seborreic keratosis, actinic keratosis, photoaged skin, and skin spots, skin cancer, systemic lupus erythematosus, psoriasis, smooth muscle cell proliferation and intimal thickening following vascular injury, inflammation, arthritis, side effects of chemotherapy, benign prostatic hyperplasia (BPH or prostate enlargement), as well as less common diseases wherein their incidence is higher in elderly people than in young people.

It is expected that any NGIP can be used in the method of the invention. In various embodiments, the NGIP is an agent that induces p53 by blocking the interaction of p53 with other proteins such as Mdm-2, FAK, COP1 and p73/p63. Thus, in one embodiment, the NGIP is an Mdm (Hdm2)-binding agent or Mdm-2 antagonist. In various embodiments, the Mdm-binding agent is a nutlin, including nutlin-3A and its analogs. In one embodiment, the NGIP is nutlin-3A. Such agents may also be used as anti-hypertrophic agents.

It is also expected that any inhibitor of mTOR can be used in the invention. The inhibitor of mTOR may be any compound that is a direct or indirect inhibitor of mTOR. Suitable indirect inhibitors of mTOR include but are not limited to Mek inhibitors, PI-3K inhibtors or AMPK activators. In one embodiment, an mTOR inhibitor is used with an NGIP.

In one embodiment, the mTOR inhibitor is rapamycin or a rapamcyin analog. Suitable rapamycin analogs include but are not limited to everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, 42-O-(2-hydroxy)ethyl rapamycin, and combinations thereof. The invention may also be performed using combinations of NGIPs and anti-hypertrophic agents.

For use in prophylaxis and/or therapy of aging related diseases, compositions described herein can be administered in a conventional dosage form prepared by mixing with a standard pharmaceutically acceptable carrier according to known techniques. Some examples of pharmaceutically acceptable carriers can be found in: Remington: The Science and Practice of Pharmacy (2005) 21st Edition, Philadelphia, Pa. Lippincott Williams & Wilkins. In various embodiments, the compositions may be provided as pharmaceutical preparations, examples of which include but are not limited to pills, tablets, mixtures, solutions, creams, liniments, eye drops, and nanoparticle compositions.

Various methods known to those skilled in the art may be used to introduce the compositions of the invention to an individual and/or in an in vitro setting. Suitable methods for administering the compositions to an indivdival include but are not limited to intracranial, intrathecal, intradermal, intramuscular, intraperitoneal, intravenous, subcutaneous, oral, intranasal and retrograde routes.

It will be recognized by those of skill in the art that the form and character of the particular dosing regime employed in the method of the invention will be dictated by the route of administration and other well-known variables, such as rate of clearance, the size of the individual and the stage of the particular disease being treated. Based on such criteria, one skilled in the art can determine an amount of any of the particular compositions described herein that will be effective for prophylaxis and/or therapy of age related diseases and/or for cellular hypertrophy in any particular individual.

The method of the invention can be performed in conjunction with conventional anti-aging and/or age-related disease therapies. The compositions of the invention could be administered prior to, concurrently, or subsequent to performing the conventional anti-aging and/or age-related disease therapies. Such therapies can include but are not limited to chemotherapies, radiation therapy and surgical interventions in the case of cancers. Further, additional compounds may be administered in conjunction with administration of the compositions according to the invention. For example, a composition comprising the NPIG could be administered with a second compound intended to augment, supplement, or provide a synergistic effect when combined with the NPIG. Such compounds include but are not limited to vitamin D, vitamin E, vitamin A, metformin, antioxidants, resveratrol, a non-steroid anti-inflammatory drug, such as a COX inhibitor, mTOR inhibitors, L-carnitine, lipoic acid, leptine, Pgp inhibitor, caspase inhibitors, and combinations thereof. Likewise, if an anti-hypertrophic compound is administered, it can be administered with a second compound intended to augment, supplement, or provide a synergistic effect when combined with the anti-hypertrophic compound. Such compounds include but are not limited to vitamin D, metformin, antioxidants, vitamins, resveratrol, non-steroid anti-inflammatory drug, such as COX inhibitors, an inhibitor of Pgp/MRP (for neurodegeneration, to decrease excretion and to change bioavailability) and inhibitors of metabolizing enzymes, and combinations of the foregoing.

The additional compounds that can be used in conjunction with the compositions comprising the NPIG and/or the anti-hypertrophic compound can be administered simultaneously, before, or after the administration of the composition comprising the NPIG and/or the anti-hypertrophic compound.

The following Examples are intended to illustrate but not limit the invention.

### EXAMPLE 1

The following Materials and Methods were used to obtain the data and results presented in this Example.

### Methods

Cell lines and reagents. HT-p21-9 and HT-p21-a cells are derivatives of HT1080 human fibrosarcoma cells, where p21 expression can be turned on or off using isopropyl-thio-galactosidase (IPTG) (7, 16, 28, 29, 36). HT-p21-9 cells express GFP, whereas HT-p21-a cells do not. HT-p16 cells are derivatives of HT1080 cells in which p16 expression can be turned on or off using IPTG (16, 36). WI-38-Tert, WI-38 are fibroblasts immortalized by telomerase. HT-p21-GSE56 cells: p53 inhibiting peptide GSE56 (18) was introduced into HT1080 p21-9 cells via a retroviral vector LXSE (37). Cells were grown in high glucose DMEM with 10% FC2 serum. WI-38-tert cells were grown in low glucose DMEM with 10% FCS. Rapamycin was obtained from LC Laboratories (Woburn, Mass.). IPTG (final concentration of 50 μg/ml) and FC2 were obtained from Sigma-Aldrich (St. Louis, Mo.). Nutlin-3a and -b were obtained from Sigma-Aldrich and La Roche, Nutley, N.J. (38). p53, p21 and p53-GFP expressing adenoviruses (Ad-p53, Ad-p21 and Ad-p53-GFP) were described previously (20, 39) and obtained from Dr. Wafik El-Deiry (Univ. Penn. Philadelphia, Pa.).

Colony formation assay. Plates were fixed and stained with 1.0% methylene blue or with crystal violet (13).

Immunoblot analysis. Proteins were separated on 4-15% gradient Tris-HCl gels (Bio-Rad). The following antibodies were used: mouse anti-actin from Santa Cruz Biotechnology, rabbit anti-phospho-S6 (Ser240/244) and (Ser235/236), mouse anti-S6, mouse anti-phospho-p70 S6 kinase (Thr389), mouse anti-p21 and anti-p53, rabbit anti-phospho-4E-BP1 (Thr37/46) from Cell Signaling; mouse anti-4E-BP1 from Invitrogen, mouse anti-p53 (Ab-6) from Calbiochem.

Beta-galactosidase staining Beta-gal staining was performed using Senescence-galactosidase staining kit (Cell Signaling Technology).

Metabolic labeling. HT-p21-9 cells were seeded at 25,000 cells/well in 12-well plates. On the next day, cells were treated with drugs. After 24 h, cells were labeled with 30 μCi [³⁵S]methionine/cysteine (Amersham) per ml of Met/Cys-free Dulbecco's modified Eagle's medium (Invitrogen) for 1 h at 37° C. Cells were washed with PBS and lysed in 1% SDS, with 0.5% BSA. To determine ³⁵S incorporation, total protein was precipitated with 0.5 ml 10% TCA and collected on nitrocellulose filters. Filters were air-dried and counted using liquid scintillation counter.

Using the Materials and Methods discussed above, the following results were obtained.

### Results

The p53 Activator Nutlin-3a Suppresses p21-Induced Senescence

Induction of p21 in HT1080-derived HT-p21-9 cells carrying an IPTG-inducible p2lexpression construct causes senescence. In the same cells, induction of p53 by nutlin-3a caused reversible cell cycle arrest (quiescence) and cells resumed proliferation after removal of nutlin-3a (Huang B, Deo D, Xia M ,Vassilev L T (2009) Pharmacologic p53 Activation Blocks Cell Cycle Progression but Fails to Induce Senescence in Epithelial Cancer Cells. Mol Cancer Res. 7: 1497-509). We used nutlin-3a, an inhibitor of p53-Mdm2 binding, in these experiments since it induces p53 at physiological levels without DNA damage and is highly specific (17). Thus, physiological levels of p53 induced quiescence, whereas ectopic expression of p21 induced senescence (Huang et al. 1999). There are two alternative models that could explain these results. First, the conventional model suggests that the physiological levels of p53 induced by nutlin-3a are not sufficient to induce p21 to the extent required to activate the senescent program in this cell line. Then addition of nutlin-3a to IPTG may only intensify senescence. A second, alternative model is that p53 actually suppresses senescence. In this case, activation of p53 by nutlin-3a in concert with IPTG-mediated induction of p21 would be expected to convert senescence into quiescence.

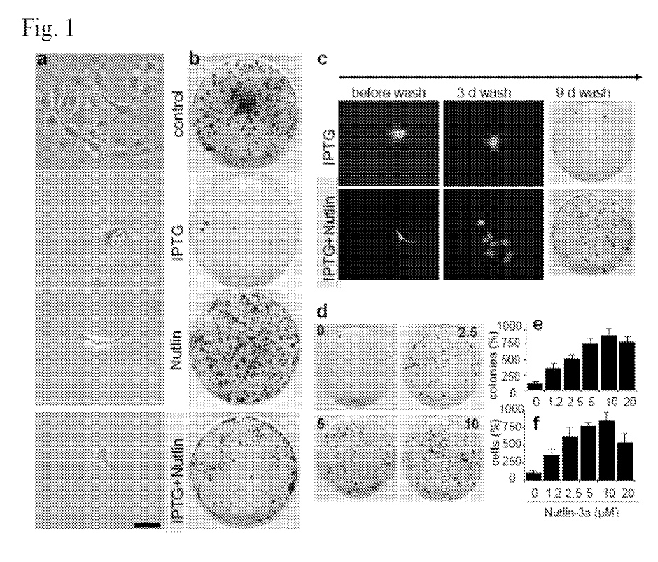

As shown in FIG. 1 and reported previously (Huang et al., 1999), IPTG- and nutlin-3a-treated cells are positive controls for senescence and quiescence, respectively. IPTG treatment induced characteristic senescent morphology (large, flat, SA-beta-Gal-positive cells), while nutlin-3a treated cells remained small, lean and SA-beta-Gal-negative (FIG. 1A). In addition, colony formation assays showed that IPTG treatment resulted in irreversible loss of proliferative potential (only a few cells formed colonies upon removal of IPTG), while nutlin-3a treatment caused reversible arrest (substantial colony formation upon nutlin-3a removal) (FIG. 1B).

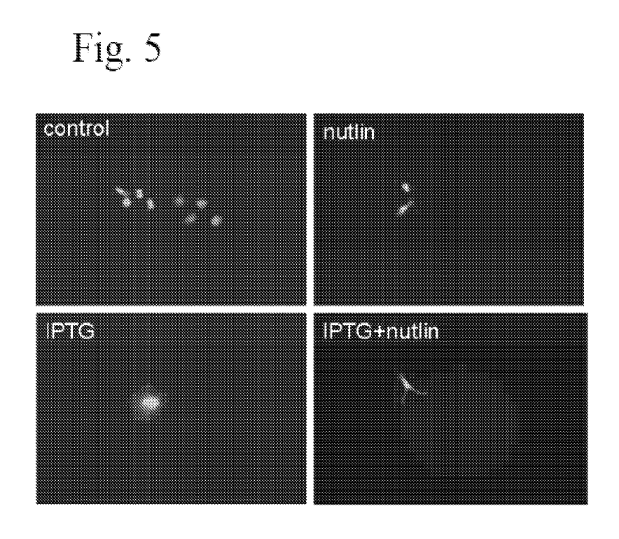

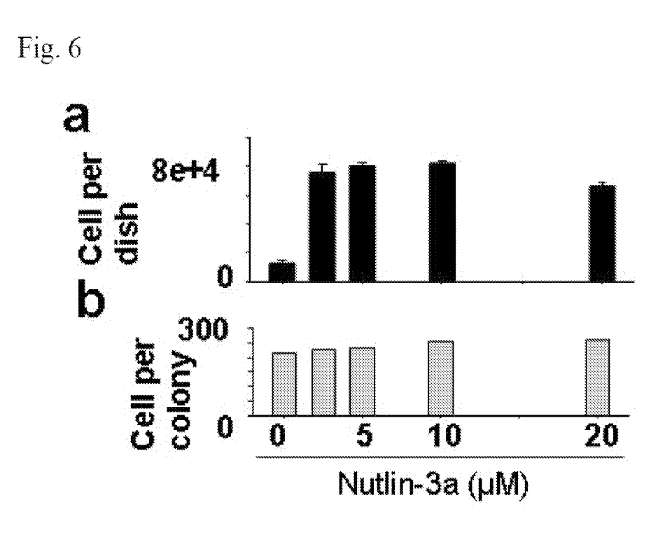

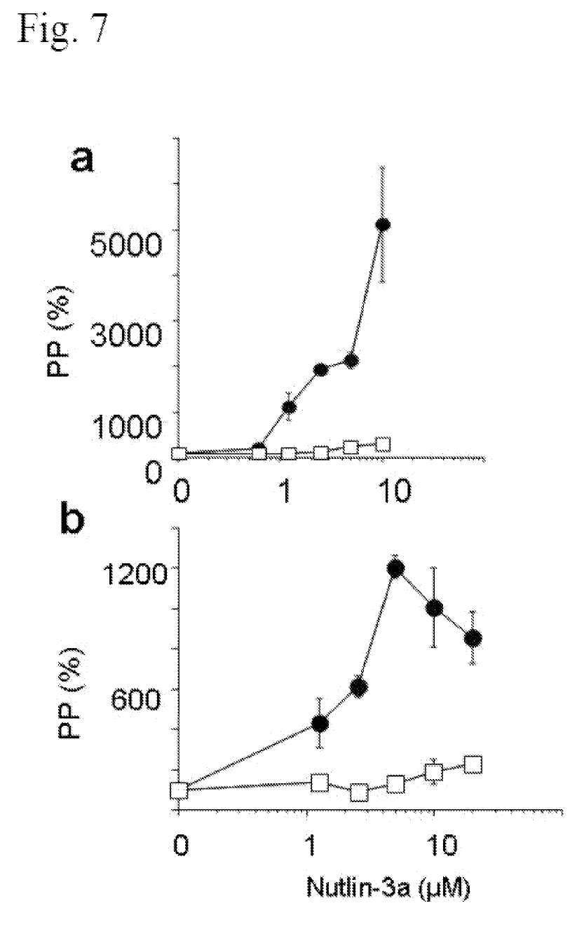

In analyzing these observations, we investigated whether addition of nutlin-3a to IPTG converts senescence into quiescence. The result of this key experiment showed that treatment with nutlin-3a prevented the senescent morphology caused by IPTG: cells remained small, lean and negative for SA-beta-Gal-staining (FIG. 1A). Furthermore, such cells retained the proliferative potential and clonogenicity (FIG. 1B). Thus we determined the effect of nutlin-3a on IPTG-induced senescence was dominant. Importantly, nutlin-3a neither abrogated nor diminished the levels of p21 (see immunoblots in Figures presented with this Example). Nutlin-3a did not abrogate the cytostatic effect of IPTG, and IPTG caused instant cell cycle arrest, manifested as solitary cells with senescent morphology at low cell density (FIG. 5). In the presence of nutlin-3a alone, cells typically underwent one division and did not proliferate further, as illustrated by colonies of 2 adjusted cells with non-senescent morphology (FIG. 5). In the presence of both nutlin-3a and IPTG, cells were arrested immediately without a single division, but did not acquire senescent morphology (FIG. 5). Thus, without abrogating cell cycle arrest caused by IPTG, nutlin-3a converted senescence into a reversible condition (quiescence). When IPTG and nutlin-3a were washed out of the cultures, the cells resumed proliferation, forming micro-colonies (FIG. 1c) and then macro-colonies (FIG. 1c). These results indicate that nutlin-3a prevented cells from undergoing IPTG-induced senescence. Suppression of senescence by nutlin-3a was observed at a range of active concentrations of nutlin-3a in a dose dependent manner (FIG. 1d-e). The most quantitative way to measure preservation of proliferative potential (PP) is the total cell number per dish. Nutlin-3a preserved proliferative potential (PP) in a dose-dependent manner (FIG. 1f). We have measured the number of cells per colony versus the number of colonies per dish (FIG. 6). Thus, nutlin-3a increased the number of cells with normal PP. The preservation of proliferative potential by nutlin-3a in IPTG-arrested cells was confirmed in both IPTG-regulated p16- and p21-expressing cells (FIG. 7).

### Suppression of Senescence Requires the Transactivation Function of p53

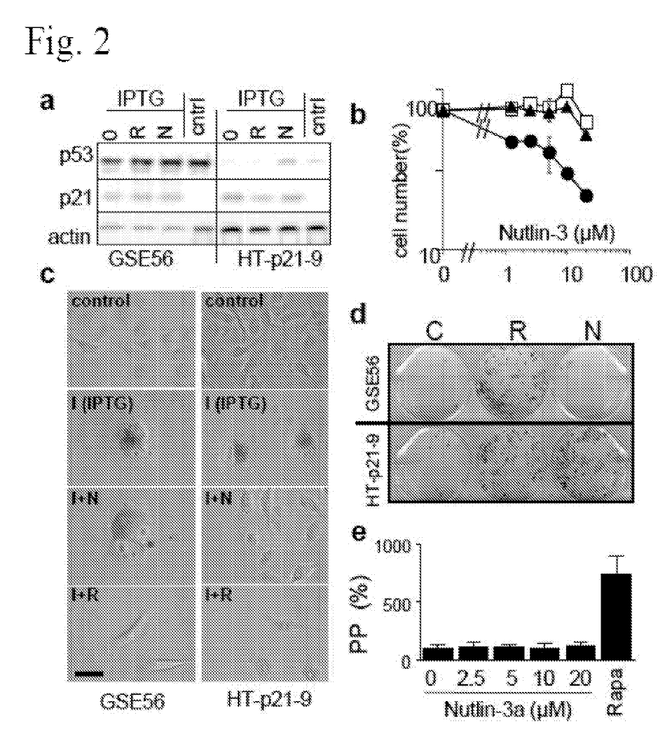

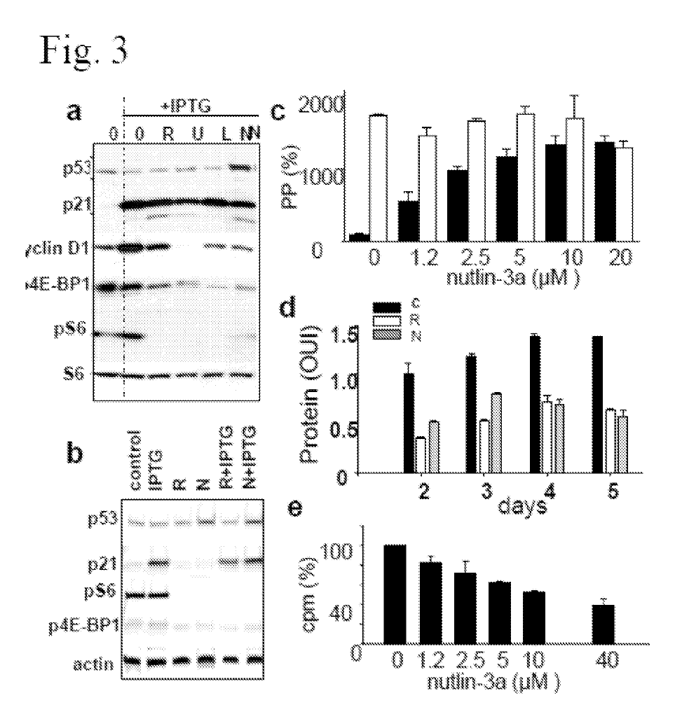

Nutlin-3a is a highly specific activator of p53 and it is believed no off-target effects of the compound have been reported. In fact, nutlin-3b, an optimer of nutlin-3a that does not block Mdm-2/p53 interaction, was not able to convert senescence into quiescence (FIG. 7b-c). To directly test whether nutlin-3a inhibits senescence by a p53-dependent mechanism, we used HT-p21-GSE56 cells, a derivative of the HT-p21cell line in which p53 function is blocked by a transdominant inhibitor, GSE56 (Ossovskaya V S, et al. (1996) Use of genetic suppressor elements to dissect distinct biological effects of separate p53 domains. Proc Natl Acad Sci US A 93: 10309-14.). Our results show that p53 was expressed at very high levels in these cells since inhibition of its transactivation function results in stabilization of the protein (analogous to mutant p53). While nutlin-3a induced p53 in HT-p21 cells, it did not affect p53 levels in HT-p21-GSE56 cells (FIG. 2a). IPTG strongly induced p21 in HT-p21-GSE56 cells and nutlin-3a did not affect this induction (FIG. 2a). Nutlin-3a failed to inhibit proliferation of HT-p21-GSE56 cells (FIG. 2b), thereby confirming that the model was adequate for testing whether suppression of senescence by nutlin-3a depends on p53. In addition, it was important to employ a positive control for p53-independent suppression of senescence. We have demonstrated that activation of mTOR (mammalian Target of Rapamycin) was required for cellular senescence, and deactivation of mTOR by rapamycin prevented senescence, causing quiescence instead. Rapamycin did not induce p53 (FIG. 2a) in agreement with its p53-independent inhibition of mTOR. Rapamycin suppressed IPTG-induced senescence in HT-p21-GSE56 cells (FIG. 2c). In contrast, nutlin-3a suppressed senescence in IPTG-treated HT-p21-9 cells only and not in similarly treated HT-p21-GSE56 cells (FIG. 2c). Consistent with these findings, nutlin-3a (unlike rapamycin) did not preserve colony formation and proliferative potential (PP) in IPTG-treated HT-p21-GSE56 cells lacking functional p53 (FIG. 2d-e). These data demonstrate that the transcriptional activity of p53 is required for suppression of senescence by nutlin-3A. In contrast, rapamycin inhibited senescence without relying on p53, as illustrated by its ability to prevent senescent morphology (FIG. 2c) and to preserve proliferative potential (FIG. 3d-e) in IPTG-treated HT-p21-GSE56 cells.

Inhibition of the mTOR Pathway by Nutlin-3a

We previously reported that inhibitors of mTOR (rapamycin), PI-3K (LY294002) and MEK (U0126) all deactivate the mTOR pathway in HT-p21-9 cells, as measured by lack of phosphorylation of the S6 ribosomal protein, and suppress cellular senescence (Demidenko Z N, Shtutman M, Blagosklonny M V (2009) Pharmacologic inhibition of MEK and PI-3K converges on the mTOR/S6 pathway to decelerate cellular senescence. Cell Cycle 8: 1896-900). Like all of these agents, nutlin-3a inhibited S6 phosphorylation and partially inhibited phosphorylatation of 4E-BP1, another downstream target of the mTOR pathway (FIG. 3 a). Nutlin-3a also normalized elevated levels of cyclin D1, associated with cellular senescence. Like rapamycin, nutlin-3a inhibited the mTOR pathway both in the presence and absence of IPTG and did not prevent induction of p21 by IPTG (FIG. 3b). Importantly, IPTG-induced p21 did not affect S6 and 4E-BP1 phosphorylation (FIG. 3a-b).

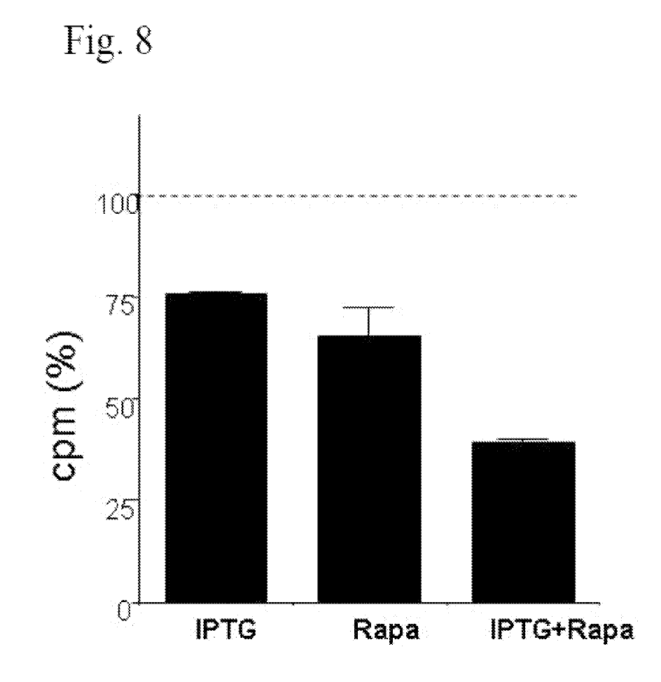

Rapamycin and nutlin-3a were equally potent in suppression of senescence (preservation of proliferative potential) in IPTG-treated HT-p21-9 cells (FIG. 3c). Moreover, in the presence of rapamycin at doses that completely inhibit mTOR, nutlin-3a could not further suppress senescence, as measured by preservation of proliferative potential (PPP) of IPTG-arrested cells (FIG. 3c). This supports the notion that nutlin-3a and rapamycin affect either the same or overlapping pathways. The mTOR pathway stimulates protein synthesis. Importantly, protein synthesis remained high in IPTG-arrested cells and is inhibited by rapamycin (FIG. 8), thus explaining cellular hypertrophy associated with senescence. Both nutlin-3a and rapamycin decreased the protein content per cell in IPTG-treated HT-p21-9 cells (FIG. 3d). To evaluate whether this decrease involved inhibition of protein synthesis, we measured ³⁵S-methionine/cysteine incorporation into nascent proteins in the presence of nutlin-3a (FIG. 3e). Nutlin-3a inhibited ³⁵S-methionine/cysteine incorporation in IPTG-treated HT-p21-9 cells in a dose-dependent manner (FIG. 3e).

### Suppression of Senescence by Ectopic Expression of p53

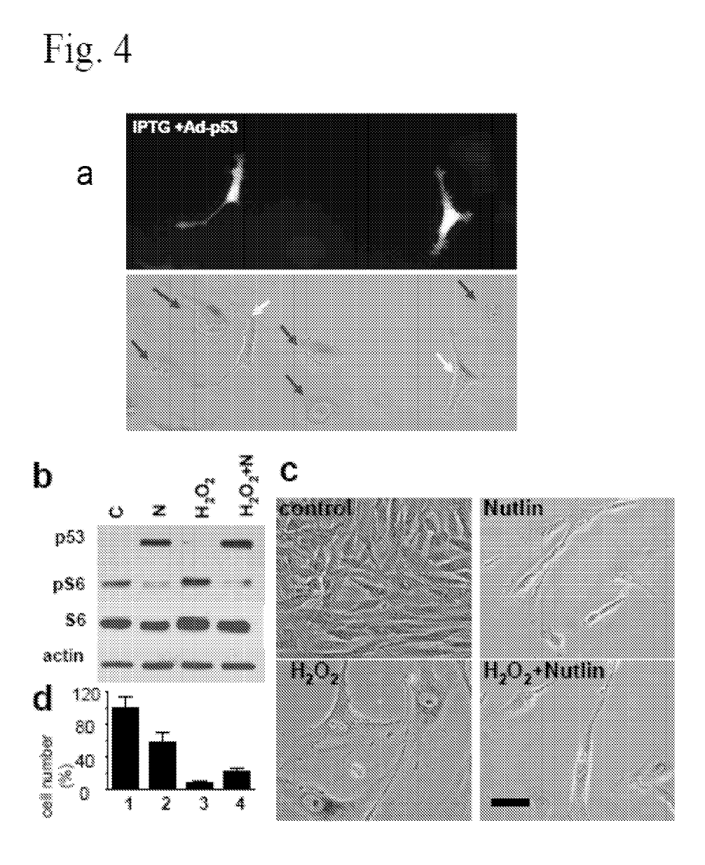

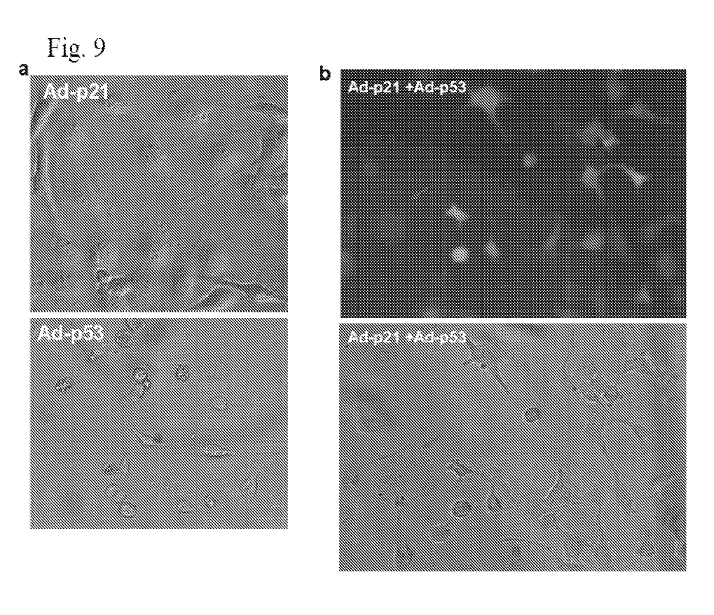

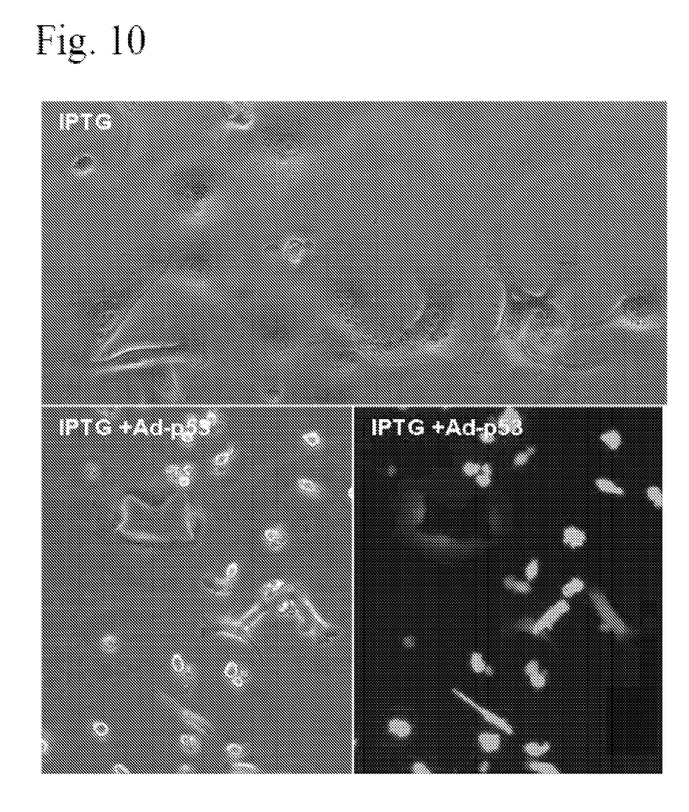

In order to confirm our results without reliance on nutlin-3a to activate p53, we tested whether expression of exogenous p53 would also lead to suppression of p21-induced senescence. We used an adenovirus that directs constitutive expression of p53 along with GFP (Ad-p53-GFP) (Wang W, Takimoto R, Rastinejad F, El-Deiry W S (2003) Stabilization of p53 by CP-31398 inhibits ubiquitination without altering phosphorylation at serine 15 or 20 or MDM2 binding. Mol Cell Biol. 23: 2171-2181.) such that infected cells can be easily identified by fluorescence microscopy. In these experiments, we used HT-p21-a cells that unlike HT-p21-9, do not express internal GFP and therefore are not green. At low titers, Ad-p53-GFP infected ˜20% of HT-p21-a cells; therefore, we were able to compare p53-overexpressing and non-infected cells on the same slide. As expected, in non-infected cells, IPTG treatment caused senescent morphology (FIG. 4a, red arrows in bottom panel). In contrast, Ad-p53-GFP-infected cells did not acquire senescent morphology (FIG. 4a). To test a different means of inducing senescence, we used infection with a p21-expressing adenovirus (Ad-p21) rather than IPTG to induce p21. Ad-p21 infected cells rapidly acquired senescent morphology, whereas Ad-p53-GFP infected cells did not (FIG. 9a). Furthermore, Ad-p53-GFP suppressed senescence caused by Ad-p21 FIG. 9b) and by IPTG-induced p16 (FIG. 10).

### Suppression of Stress-Induced Senescence in Fibroblasts

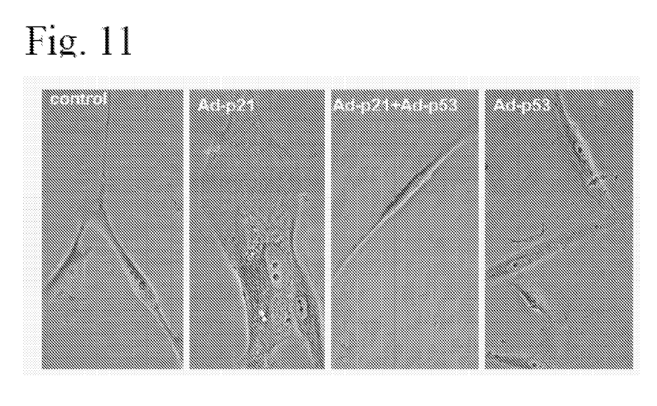

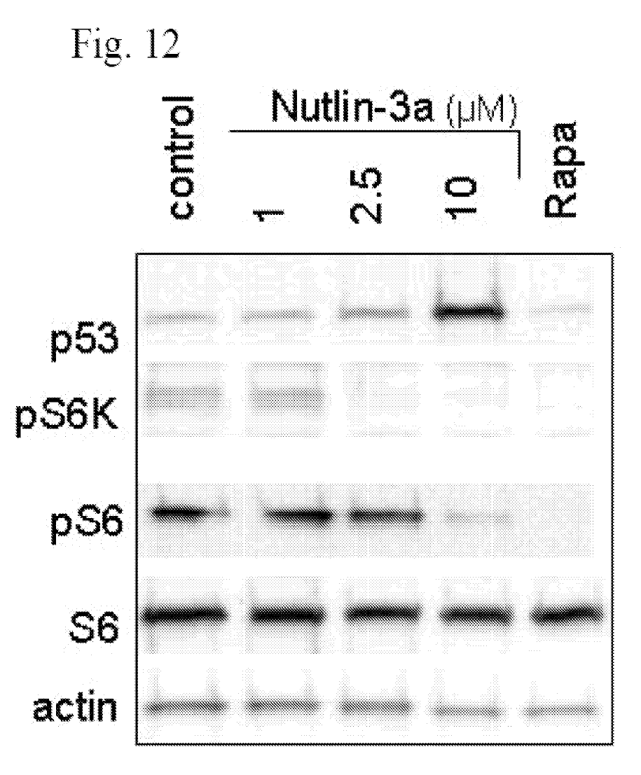

To extend our observation of p53-mediated suppression of senescence to cells unrelated to HT1080, we used telomerase-immortalized human WI-38 fibroblasts (WI-38-tert cells). As shown in Supplemental FIG. 11, infection of these cells with Ad-p53 also resulted in quiescent morphology (slim, beta-Gal-negative cells); however, infection with Ad-p21 induced senescent morphology. Most importantly, co-infection of the cells with Ad-p53 and Ad-p21 demonstrated that p53 suppressed p21-induced senescence (FIG. 11). Since Ad-p53 infection resulted in excessive levels of p53, the observed effect was limited by concomitant induction of apoptosis. Therefore, we used nutlin-3a to induce p53 at physiological levels in this system. We demonstrated that treatment of WI-38-tert cells with nutlin-3a caused quiescence. Importantly, nutlin-3a (at concentrations that induce p53) inhibited S6K and S6 phosphorylation (FIG. 12). In contrast, doxorubicin does not inhibit mTOR. This may explain why nutlin-3a induced quiescence in WI-38-tert cells, whereas doxorubicin caused senescence in WI-38-tert cells. We next investigated whether nutlin-3a could suppress senescence caused by hydrogen peroxide (H₂O₂), a canonical inducer of cellular senescence in fibroblasts. In WI-38-tert cells, H₂O₂ inhibited cell proliferation without induction of p53 and without affecting S6 phosphorylation (FIG. 4 b). This results in senescent morphology (FIG. 4c). Nutlin-3a induced p53, inhibited S6 phosphorylation (FIG. 4b) and suppressed senescence induced by H₂O₂ (FIG. 5c). Furthermore, nutlin-3 partially preserved proliferative potential in H₂O₂-treated cells (FIG. 4d). Thus, we have used different cell lines, as well as various means of inducing cellular senescence and of activating p53, to demonstrate that p53 suppresses senescence.

Thus, it will be recognized from the foregoing that it is disclosed herein for the first time that p53-induced quiescence actually results from suppression of senescence by p53.

### EXAMPLE 2

The following Materials and Methods were used to obtain the results disclosed in this Example.

### Materials and Methods

Cell lines and reagents. In HT-p21 cells, p21 expression can be turned on or off using isopropyl-thio-galactosidase (IPTG) [14, 15]. HT-p21 cells were cultured in DMEM medium supplemented with FC2 serum. Rapamycin was obtained from LC Laboratories and dissolved in DMSO as 2 mM solution and was used at final concentration of 500 nM, unless otherwise indicated. IPTG and FC2 were obtained from Sigma-Aldrich (St. Louis, Mo.). IPTG was dissolved in water as 50 mg/ml stock solution and used in cell culture at final concentration of 50 μg/ml.

Immunoblot analysis. Cells were lysed and soluble proteins were harvested as previously described [9]. Immunoblot analysis was performed using mouse monoclonal anti-p21, mouse monoclonal anti-phospho-S6 Ser240/244 (Cell Signaling, MA, USA), rabbit polyclonal anti-S6 (Cell Signaling, MA, USA) and mouse monoclonal anti-tubulin Ab as previously described [9]. Cell counting. Cells were counted on a Coulter Z1 cell counter (Hialeah, Fla.). Colony formation assay. Two thousand HT-p21 cells were plated per 100 mm dishes. On the next day, cells were treated with 50 μg/ml IPTG and/or 500 nM rapamycin, as indicated. After 3 days, the medium was removed; cells were washed and cultivated in the fresh medium. When colonies become visible, plates were fixed and stained with 0.1% crystal violet (Sigma). Plates were photographed and the number of colonies were determined as previously described [9]. SA-Gal staining Cells were fixed for 5 min in beta-galactosidase fixative (2% formaldehyde; 0.2% glutaraldehyde in PBS), and washed in PBS and stained in-galactosidase solution (1 mg/ml 5-bromo-4-chloro-3-indolyl-beta-gal (X-gal) in 5 mM potassium ferricyamide, 5 mM potassium ferrocyamide, 2 mM MgCl₂ in PBS) at 37° C. until beta-Gal staining become visible in either experiment or control plates. Thereafter, cells were washed in PBS, and the number of -galactosidase activity-positive cells (blue staining) were counted under bright field illumination.

Using the Materials and Methods described above for this Example, the following results were obtained.

### Exponential Mass-Growth Precedes Senescence

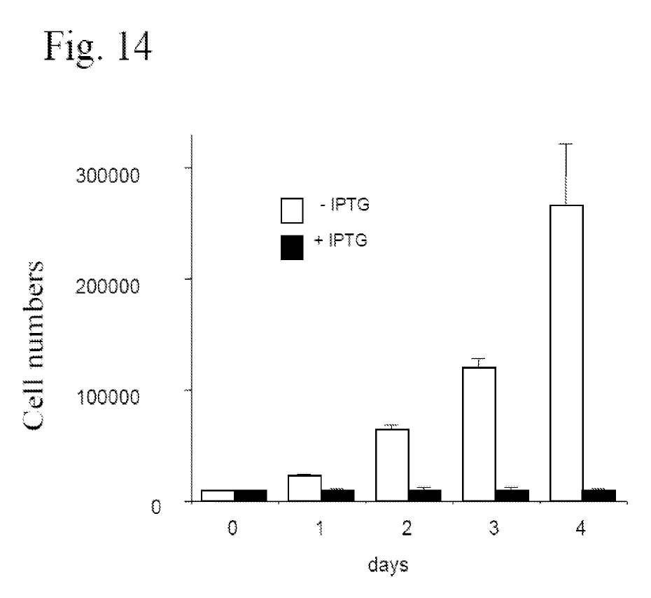

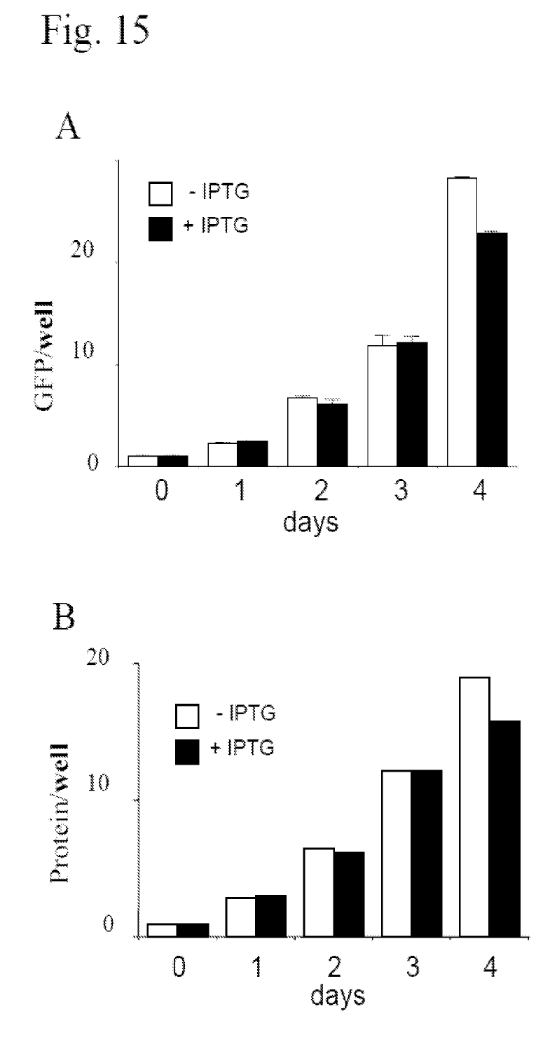

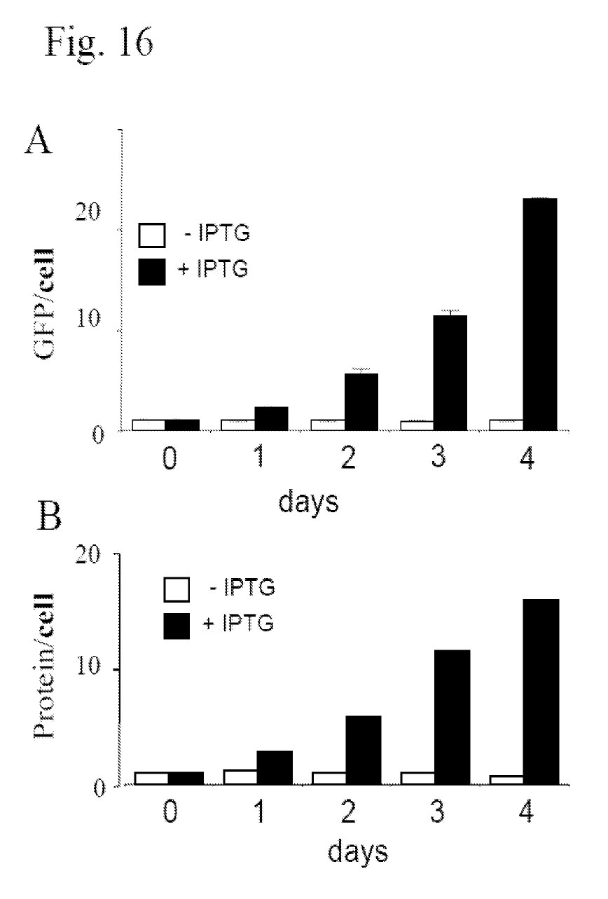

A number of proliferating cells increased exponentially (with a doubling time 20-24 h). As in Example 1, induction of p21 by IPTG caused G1 and G2 arrest, completely blocking cell proliferation (FIG. 14). p21-arrested cells continued to grow in size, becoming hypertrophic. Since the cells contained CMV-driven EGFP, we measured both protein and GFP. Per well, amounts of GFP and protein were increased almost exponentially with or without IPTG (FIG. 15). Per cell, amounts of GFP and protein were increased only for IPTG-treated (non-dividing) cells (FIG. 16). For proliferating cells (no IPTG), GFP per cell and protein per cell remained constant (FIG. 16), because mass growth was balanced by cell division. In contrast, in IPTG-treated cells, protein/cell and GFP/cell increased almost exponentially for 3 days (FIG. 16). During induction of senescence by IPTG, cellular mass continued to increase but was not balanced by cell division. In all cases, protein and GFP correlated (FIG. 16), making GFP per cell a convenient marker of cellular hypertrophy.

These data can explain how induction of p21 can induce GFP without trans-activating CMV promoter: by inhibiting cell cycle without inhibiting cell growth. Furthermore, the notion that GFP per cell is a marker of hypertrophy yields 2 predictions. First, mutant p21 that cannot bind CDKs and thus cannot arrest cell cycle will not induce GFP. Second, anti-hypertrophic agents such as rapamycin will reduce GFP per cell without abrogating cell cycle arrest.

### Dose Dependent Suppression of Cellular Hypertrophy

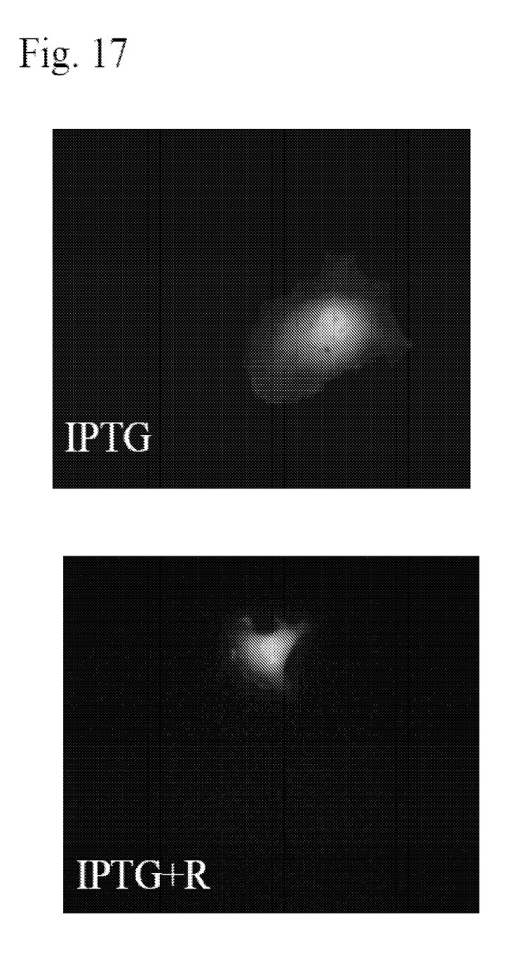

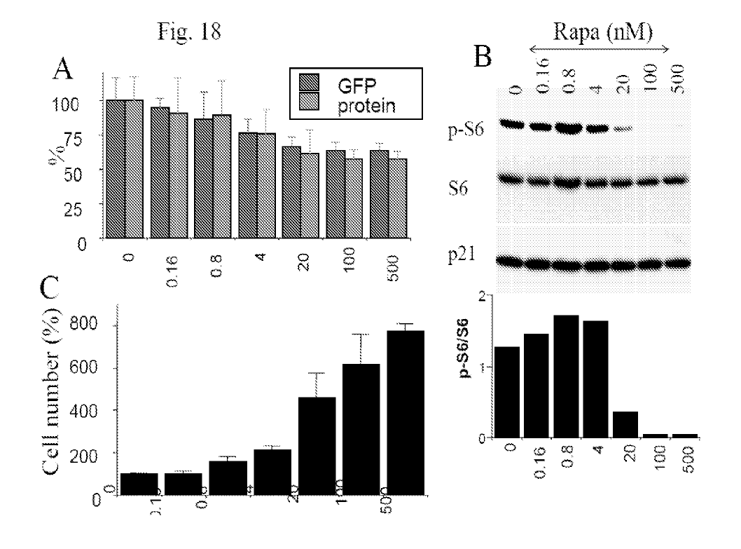

We next investigated the effects of rapamycin on hypertrophy of senescent cells. Cells were induced to senesce by IPTG in the presence (+R) or the absence of rapamycin. On days 3 and 5 effects of rapamycin on cellular hypertrophy were evaluated. By microscopy, the anti-hypertrophic effect of rapamycin was the most evident at low cell densities (such as 1000 cells per 60-mm dish) because there was a sufficient space for IPTG-treated cells to grow in size in the absence of rapamycin (FIG. 17). However, we could not reliably measure protein levels at such low cell densities. At regular cell densities, rapamycin (500 nM) reduced cellular hypertrophy by 30%-40% (FIG. 18A and data not shown). Two markers of hypertrophy (protein/cell and GFP/cell) correlated (FIG. 18A). The anti-hypertrophic effect of rapamycin was not statistically significant at concentrations of rapamycin below 20 nM. At first, this was puzzling given that rapamycin inhibits the mTOR pathway at low concentrations in many cell types. Therefore, we investigated a dose response of mTOR inhibition by measuring S6 phosphorylation, a marker of mTOR activity. In agreement with anti-hypertrophic effects, rapamycin inhibited S6 phosphorylation at concentrations 20 nM or higher, achieving maximal effects at 100 nM-500 nM (FIG. 18B). Thus, inhibition of S6 phosphorylation and inhibition of hypertrophy correlated, explaining the requirements of high concentration (100-500 nM) of rapamycin for anti-hypertrophic effects in this particular cell line.

### Dose-Dependent Preservation of Cellular Competence

Rapamycin preserves proliferative potential in arrested cells meaning that cells can successfully divide when the arrest is lifted. But rapamycin does not induce proliferation and in contrast can cause quiescence (in some cell types). To clearly distinguish the potential to proliferate (competence) and actual proliferation, we use the terms competence (the potential to proliferate) and incompetence (permanent loss of proliferative potential associated with cellular senescence). In HT-1080 cells, rapamycin preserves competence during cell cycle arrest caused by p21. Unlike senescent cells, quiescent cells are competent.

We determined whether preservation of competence (PC) correlated with inhibition of S6 phosphorylation and the anti-hypertrophic effect of rapamycin. Cells were treated with IPTG and increasing concentrations of rapamycin ranging from 0 to 500 nM (FIG. 18 C). After 3 days, IPTG was washed out, thus allowing the cells to proliferate, and after another 5 days cells were counted. The IPTG-treated cells became incompetent, whereas rapamycin suppressed incompetence (FIG. 18 C). Remarkably, preservation of competence was detectable at lower concentrations of rapamycin than those that inhibited either S6 phosphorylation or cellular hypertrophy. In part, such a higher sensitivity of a PC-test compared with inhibition of hypertrophy may be due to the relative magnitudes of the effects (30% inhibition of hypertrophy versus 800% PC). It is possible that even a transient inhibition of mTOR (not detected by immunoblot) detectably increased competence. Consistent with this explanation, even when rapamycin was added with delay, preservation of competence was detectable.

### Exponential Proliferation of Competent Cells

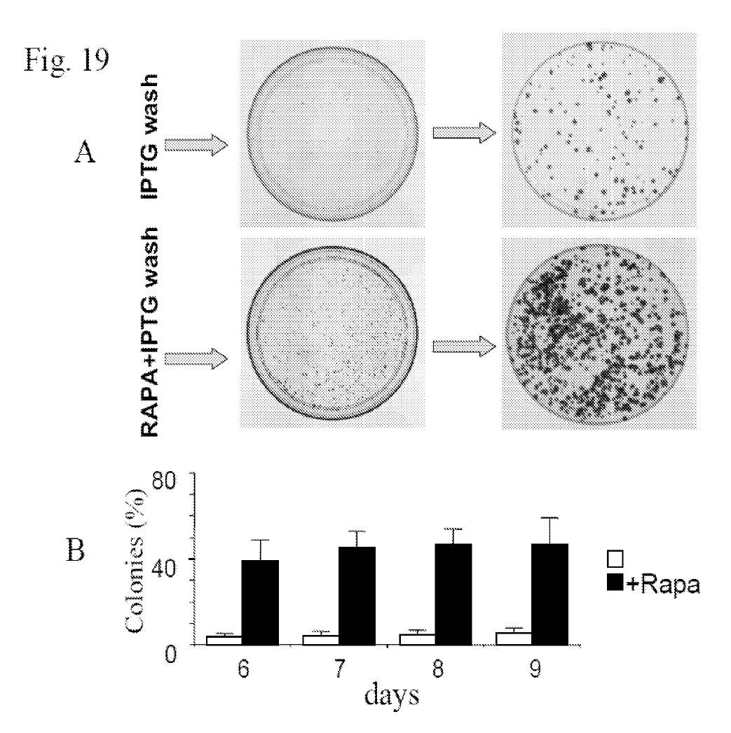

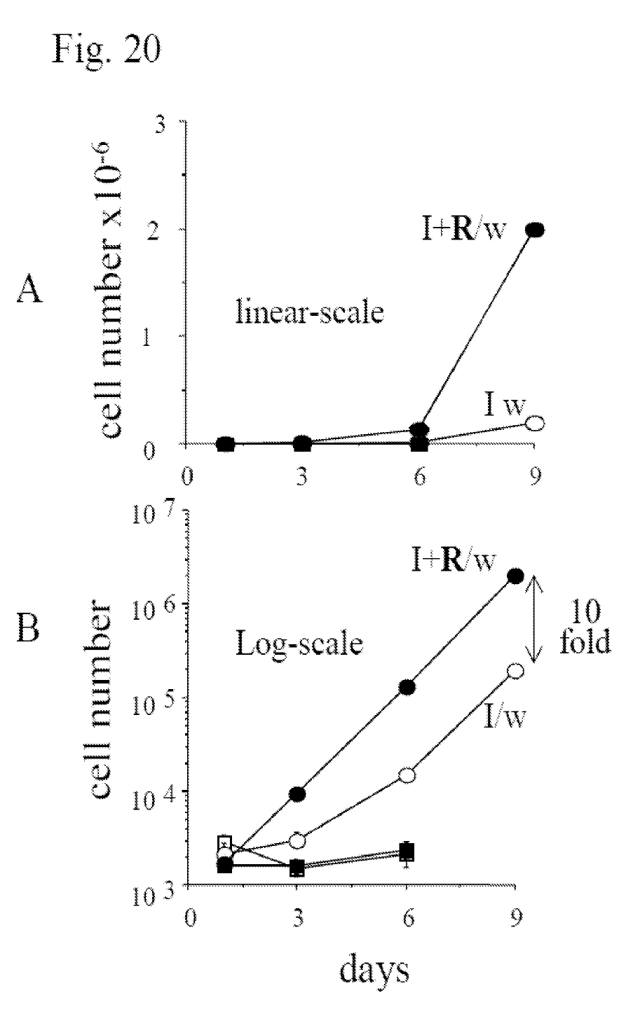

In the presence of IPTG (with or without rapamycin), the cells did not proliferate and did not form colonies. When IPTG was washed out, 3-5% cells remained competent even without rapamycin (FIG. 19). Colonies grew in size, while the number of colonies was almost unchanged (FIG. 19). Rapamycin increased a number of colonies (a number of competent cells) almost 10-fold. We further compared the proliferative quality of competent cells remained after treatment with IPTG either without or with rapamycin (I/w and I+R/w, respectively). In I/w and I+R/w conditions, the number of cells started to increase exponentially after 1 day and 3 days, respectively (FIG. 20). After 6 days, both curves (I/w and I+R/w) became parallel. The curve “I+R/w” was just shifted to the right on approximately 3 days (FIG. 20). This corresponded to a 10-fold difference in an initial number of competent cells, if their doubling time was around one day. Noteworthy, this also corresponds to the initial difference in the number of competent cells as determined by colony formation (FIG. 19). Also, both in I/w and I+R/w conditions, doubling time of the competent cells was around 20-24 hours, similar to the proliferative rate of the untreated cells.

### Reversal of Hypertrophy During Proliferation of Competent Cells

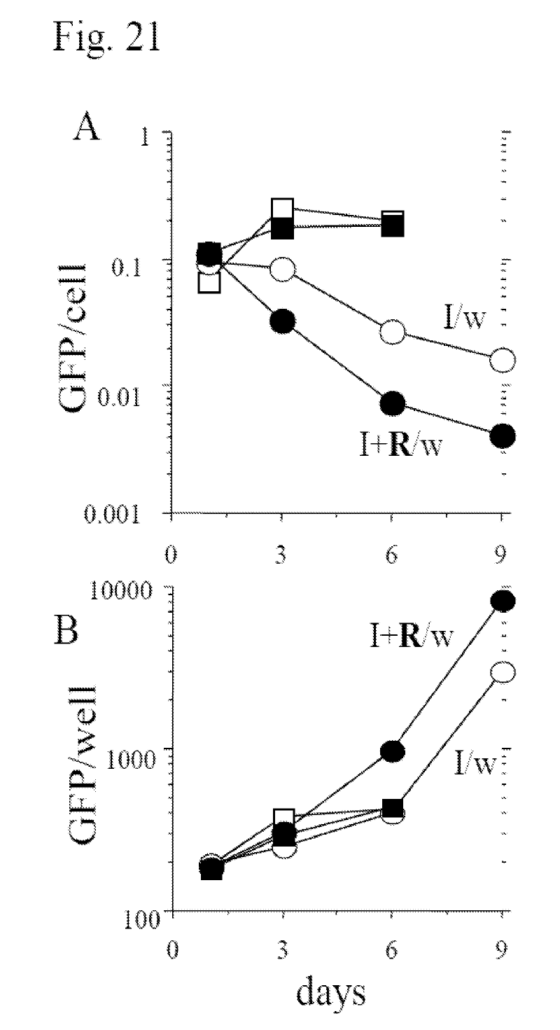

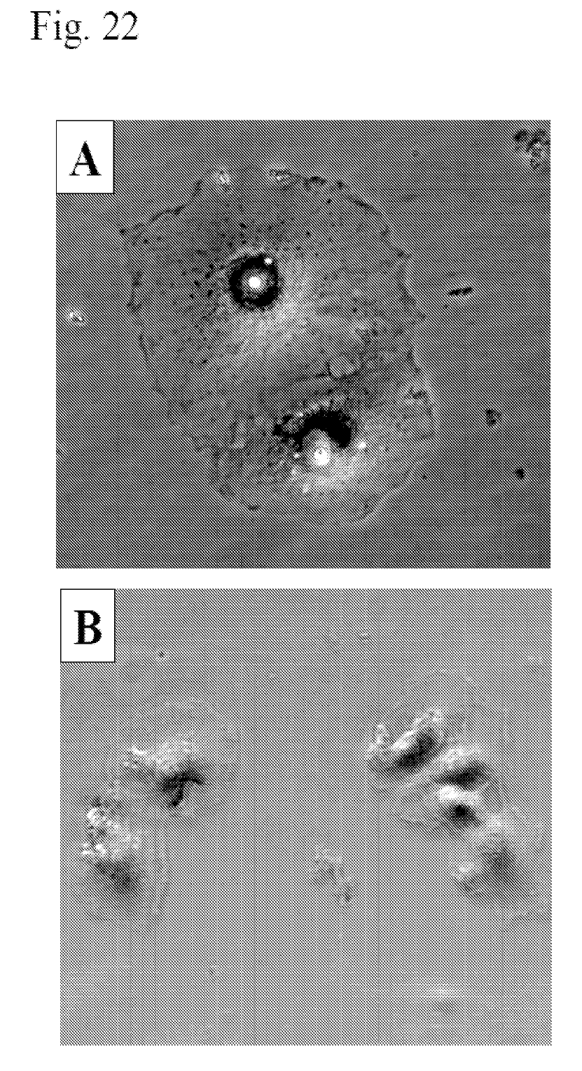

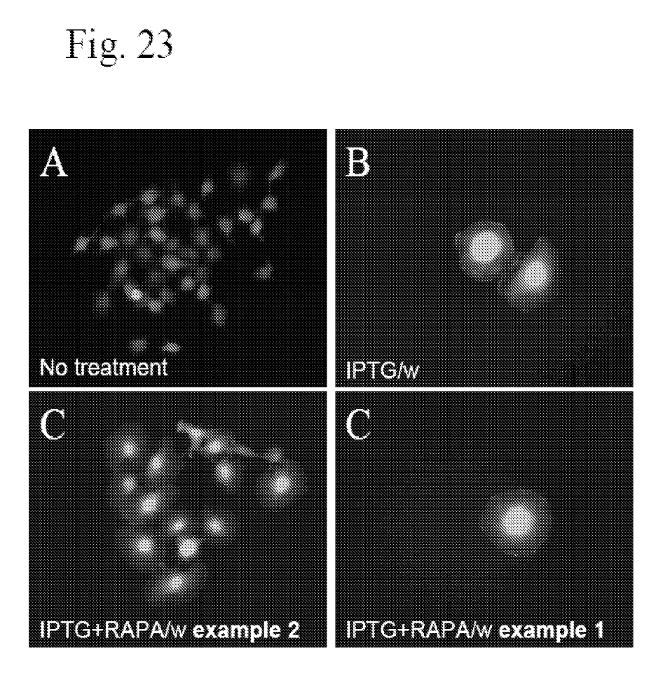

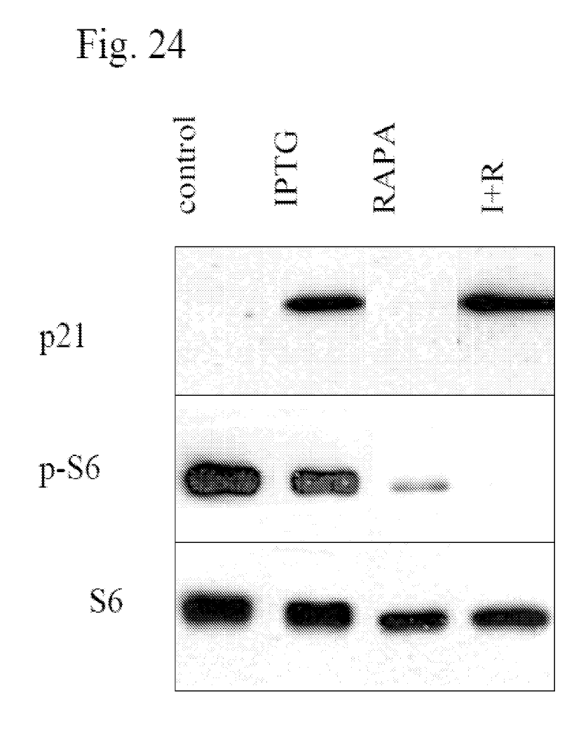

Rapamycin decreased cellular hypertrophy approximately 30% in IPTG treated cells (FIG. 18A). When IPTG and rapamycin were washed out, there was a lag period about 24-30 hrs for competent cells to undergo first division (supplementary movie will be available at). During the lag period, cells grew in size, because rapamycin was washed out. Consequently, as measured by GFP per cell (FIG. 21A), rapamycin-treated cells reached the size of the cells treated with IPTG alone (FIG. 21A: I/w and I+R/w at day one). Similarly, as measured by protein per cell, the cells treated with IPTG plus rapamycin become fully hypertrophic at day one after wash (data not shown). Despite regaining hypertrophy, IPTG+rapamycin-treated cells remained competent (FIG. 19-20). This indicates that hypertrophy was not a cause of proliferative incompetence in IPTG-treated cells. When competent cells divided, GFP per cell decreased (FIG. 21 B). In agreement, there was a marked difference in cell morphology of typical cells in both conditions (FIG. 22). Under I/w conditions, most of the cells were still large and flat, expressing beta-Gal staining Under I+R/w conditions, predominant cells were with a small-cell morphology and beta-Gal-negative. These cells formed colonies, indicating that they acquired non-senescent morphology due to proliferation (FIG. 23 C). In contrast, senescent cells that did not resume proliferation remained large (FIG. 23 C). Competent cells, while proliferating and forming colonies, became smaller in size (FIG. 23 C). Eventually, the average cell size dropped to normal levels under I+R/w conditions, coincident with a decrease in both the amount of protein/cell and GFP/cell coincided (FIG. 24), indicating that both are markers of cellular hypertrophy. Despite reversal of hypertrophy and a drop in GFP/cell, the amount of total GFP and protein per well increased due to cell proliferation (FIG. 21 B and data not shown).

We claim:

1. A method of suppression and/or deceleration of mammalian cellular aging by contacting mammalian cells with a composition comprising a non-genotoxic inducer of p53 (NGIP).

2. The method of claim 1, wherein the NGIP is nutlin, nutlin-3A, a nutlin analog, or a combination thereof

3. The method of claim 2, wherein the mammalian cells are present in a human.

4. The method of claim 3, wherein the human has not been diagnosed with cancer.

5. The method of claim 4, wherein the NGIP is nutlin-3A.

6. The method of claim 1, wherein the NGIP is a Mdm-binding agent or Mdm-2 antagonist.

7. The method of claim 4, wherein the suppression and/or deceleration of mammalian cellular aging comprises the mammalian cells becoming quiescent.

8. The method of claim 1, wherein the NGIP induces p53 by blocking p53 interaction with other proteins.

9. The method of claim 3, wherein the human is in need of prophylaxis or therapy for an age-related diseases selected from the group consisting of benign prostatic hyperplasia, angioma, cardiovascular diseases, atherosclerosis, hypertension, osteoporosis, insulin-resistance and type II diabetes, Alzheimer's disease, Parkinson's disease, age-related macular degeneration, retinopathy, systemic lupus erythematosus, psoriasis, smooth muscle cell proliferation and intimal thickening following vascular injury, inflammation, arthritis, side effects of chemotherapy, and combinations thereof.

10. A method for reducing cellular hypertrophy in an organism comprising administering a therapeutically effective amount of a composition comprising an anti-hypertrophic compound to the organism.

11. The method of claim 10, wherein the anti-hypertrophic compound is selected from the group consisting of nutlin, nutlin-3A, a nutlin analog, or a combination thereof, and wherein the organism does not have cancer.

12. The method of claim 10, wherein the anti-hypertrophic compound is rapamycin or a rapamycin analog.

13. The method of claim 12, wherein the organism is not in need of immunosuppression and has not been previously treated with the rapamycin or the rapamycin analog.

14. The method of claim 13, wherein the rapamycin analog is selected from the group consisting of everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin, and combinations thereof.

15. The method of claim 10, wherein the composition comprises a combination of nutlin-3a and rapamycin or a rapmycin analog.
